(19) United States
(12) Patent Application Publication
(10) Pub. No.: US 2017/0252487 A1
(43) Pub. Date: Sep. 7, 2017

(54) Insulin-Mimetic Composite for Bone Repair

(71) Applicant: New Jersey Institute of Technology, Newark, NJ (US)

(72) Inventors: Treena Lynne Arinzeh, West Orange, NJ (US); Svetlana Dukleska-Schussler, Elmwood Park, NJ (US)

(73) Assignee: New Jersey Institute of Technology, Newark, NJ (US)

(21) Appl. No.: 15/447,906

(22) Filed: Mar. 2, 2017

Related U.S. Application Data
(60) Provisional application No. 62/303,459, filed on Mar. 4, 2016.

(51) Int. Cl. A61L 27/54; A61L 27/38; A61L 27/46

(52) U.S. Cl. CPC A61L 27/54; A61L 27/46; A61L 27/3834; A61L 27/3821; A61L 2430/02; A61L 2300/412; A61L 2400/12; A61L 2300/224

(57) ABSTRACT

The present disclosure generally relates to a composite scaffold containing insulin-mimetic materials for healing bone defects (e.g., bone repair). In particular, the present disclosure relates to a fibrous composite containing a synthetic polymer, nanoceramic and a vanadium salt to improve the healing of bone defects.

### CROSS-REFERENCE TO RELATED APPLICATION

The present application claims priority benefit to a provisional application entitled “Insulin-Mimetic Composite for Bone Repair” which was filed on Mar. 4, 2016, and assigned Ser. No. 62/303,459. The entire content of the foregoing provisional application is incorporated herein by reference.

### FIELD OF THE TECHNOLOGY

The present disclosure generally relates to a composite scaffold containing insulin-mimetic materials for healing bone defects (e.g., bone repair). In particular, the present disclosure relates to a fibrous composite containing a synthetic polymer, nanoceramic and a vanadium salt to improve the healing of bone defects.

### BACKGROUND

Bone defects, including large bone defects, are difficult to repair properly. In the case of large bone defects, the risk of non-union of the affected bone is real. Conventional treatment methods, such as autografts and allografts, have limitations. These limitations include limited availability, donor site morbidity, potential disease transmission, and poor healing or incorporation. The present disclosure relates to an alternate treatment method for treating bone defects.

### SUMMARY

The present disclosure generally relates to a composite scaffold containing insulin-mimetic materials for healing bone defects (e.g., bone repair).

In one embodiment, the present disclosure relates to a composite scaffold capable of supporting cell and tissue growth including (i) a synthetic polymer, wherein the synthetic polymer is formed into a plurality of fibers, (ii) at least one nanoceramic, and (iii) an insulin-mimetic compound. The scaffold can contain about 0.01 to about 0.1 wt % of the insulin-mimetic compound. The scaffold can also contain whole bone marrow or isolated mesenchymal stem cells. In a particular embodiment, a vanadium compound is incorporated in a composite scaffold containing polycaprolactone (PCL) and 20/80 HA/TCP (wt %/wt %) nanoparticles.

In another embodiment, the present disclosure relates to a method for repairing a bone defect comprising the step of applying a composite scaffold to the bone defect, wherein the scaffold includes (i) a synthetic polymer, wherein the synthetic polymer is formed into a plurality of fibers, (ii) at least one nanoceramic, and (iii) an insulin-mimetic compound, wherein the insulin-mimetic compound is continually released from the scaffold. The insulin-mimetic compound can be released over about 7 to about 30 days. The insulin-mimetic compound can be released at a rate of at least about 10 ng/day.

Thus, in exemplary embodiments, the present disclosure provides a composite scaffold capable of supporting cell and tissue growth that includes (i) a synthetic polymer, wherein the synthetic polymer is formed into a plurality of fibers; (ii) at least one nanoceramic; and (iii) an insulin-mimetic compound, wherein the scaffold contains about 0.01 to about 0.1 wt % of the insulin-mimetic compound.

The disclosed composite scaffold may further include, for example, mesenchymal stem cells and/or whole bone marrow.

The synthetic polymer may include, for example, polylactic acid, poly L-lactic acid, polyglycolic acid, polylactic co-glycolic acid, poly ε-caprolactone, poly methacrylate co-n-butyl methacrylate, poly dimethyl siloxane, polyethylene oxide and combinations thereof. In exemplary embodiments, the scaffold contains about 65 to about 75 wt % of the synthetic polymer.

In exemplary embodiments, the nanoceramic(s) may include hydroxy apatite, tricalcium phosphate, biphasic calcium phosphate, calcium carbonate, calcium sulfate, bioactive glass, biphasic bioceramic and combinations thereof. For example, the nanoceramic may be a biphasic bioceramic hydroxyapatite/β-tricalcium phosphate. The composite scaffold may include about 25 to about 35 wt % of nanoceramic, and such nanoceramic may be uniformly distributed throughout the scaffold.

In exemplary embodiments, the insulin-mimetic compound may take the form of vanadium, zinc, tungsten, selenium, molybdenum, niobium, manganese compounds, and combinations thereof. In an exemplary implementation, the insulin-mimetic compound is vanadyl acetylacetonate. The scaffold may advantageously contain about 0.03 to about 0.07 wt % of the insulin-mimetic compound.

In addition, the present disclosure provides an advantageous method for repairing a bone defect that includes, inter alia, applying a composite scaffold to the bone defect, wherein the scaffold includes: (i) a synthetic polymer, wherein the synthetic polymer is formed into a plurality of fibers; (ii) at least one nanoceramic; and (iii) an insulin-mimetic compound, wherein the scaffold contains about 0.01 to about 0.1 wt % of the insulin-mimetic compound, and wherein the insulin-mimetic compound is continually released from the scaffold.

In exemplary embodiments, the insulin-mimetic compound may be advantageously released over 7-28 days, and may be released at a rate of at least about 10 ng/day. The composite scaffold may contains at least about 20% of the insulin-mimetic compound after 7 days after application. The production of collagen, GAG, osteocalcin or combinations thereof may be advantageously increased according to the disclosed method.

The present disclosure thus provides a number of advantages, including an improved method of treating large bone defects. The composite scaffold can promote osteogenic/chondrogenic activity and other key markers important for bone repair.

Additional features, functions and benefits of the present disclosure will be apparent from the description which follows, particularly when read in conjunction with the appended figures.

### BRIEF DESCRIPTION OF THE DRAWINGS

The invention will be more fully understood from the following detailed description taken in conjunction with the accompanying drawings, in which:

FIG. 1 shows increased gene expression for VEGF for cells on VAC Composite relative to Composite control as tested in Example 1. A value >1.5 is considered significant.

FIG. 2 shows an increase in total collagen (hydroxyproline) production at 28 days as tested in Example 1. (*p<0.05 between the VAC composite vs. the composite control, # p<0.05 between C/O vs. OS media).

FIG. 3 shows increased GAG production at 28 days as tested in Example 1. (*p<0.05 between the VAC composite vs. the composite control, ̂p<0.05 between CCM vs. C/O media).

FIG. 4 shows increased osteocalcin production at 28 days as tested in Example 1. (*p<0.05 between the VAC composite vs. the composite control, # p<0.05 between C/O vs. OS media).

### DETAILED DESCRIPTION

The present disclosure generally relates to a composite scaffold containing insulin-mimetic materials for healing bone defects (e.g., bone repair). In particular, the present disclosure relates to a fibrous composite containing a synthetic polymer, nanoceramic and an insulin-mimetic to improve the healing of bone defects. The scaffold can be biocompatible, biodegradable or both.

In one embodiment, the present disclosure relates to a composite scaffold capable of supporting cell and tissue growth including (i) a synthetic polymer, wherein the synthetic polymer is formed into a plurality of fibers, (ii) at least one nanoceramic, and (iii) an insulin-mimetic compound.

The synthetic polymer can be a single polymer or copolymer. Suitable polymers can include poly (α-hydroxy acids), such as the polyesters. The synthetic polymer can be selected from the group consisting of polylactic acid, poly L-lactic acid, polyglycolic acid, polylactic co-glycolic acid, poly ε-caprolactone, poly methacrylate co-n-butyl methacrylate, poly dimethyl siloxane, polyethylene oxide and combinations thereof. For example, the synthetic polymer can be poly ε-caprolactone (PCL).

The amount of synthetic polymer in the scaffold can vary depending on the properties desired. The scaffold can contain about 50, 55, 60, 65, 66, 67, 68, 69, 70, 71, 72, 73, 74, 75, 80, 85 or about 90 wt % of the synthetic polymer. These values can be used to define a range, such as about 65 to about 75 wt % synthetic polymer.

The synthetic polymer can be formed into a plurality of fibers, such as by electrospinning. The scaffold containing a plurality of fibers can have varying physical characteristics, such as porosity, pore size, interfiber spacing, fiber diameter, depending on the properties desired. In some embodiments, the scaffold can be solvent-cast.

Porosity of scaffold can be about 40, 45, 50, 55, 60, 65, 70, 75, 80, 85, 90 or about 95%. These values can be used to define a range, such as about 80 to about 85%.

Pore size of the scaffold can be about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440 or about 450 micrometers. These values can be used to define a range, such as about 180 to about 240 micrometers.

Interfiber spacing of the scaffold can be about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or about 500 micrometers. These values can be used to define a range, such as about 200 to about 260 micrometers.

The fibers can have diameters ranging from about 100 nanometers to about 100 micrometers. The fiber diameter can be about 100 nm, 200 nm, 300 nm, 400 nm, 500 nm, 600 nm, 700 nm, 800 nm, 900 nm, 1 μm, 10 μm, 20 μm, 30 μm, 40 μm, 50 μm, 60 μm, 70 μm, 80 μm, 90 μm or about 100 μm. These values can be used to define a range, such as about 900 nm to about 1 μm.

The at least one nanoceramic, or nanoparticle ceramic material, can be selected from the group consisting of hydroxy apatite, tricalcium phosphate, biphasic calcium phosphate, calcium carbonate, calcium sulfate, bioactive glass, biphasic bioceramic and combinations thereof. For example, the at least one nanoceramic can be a combination of hydroxy apatite and tricalcium phosphate, such as a biphasic bioceramic hydroxyapatite/β-tricalcium phosphate. The relative amounts of hydroxyapatite to β-tricalcium phosphate in the nanoceramic can vary. For example, the following composition (in wt %) of hydroxyapatite/β-tricalcium phosphate can be used: 15 HA/85 βTCP, 16 HA/84 βTCP, 17 HA/83 βTCP, 18 HA/82 βTCP, 19 HA/81 βTCP, 20 HA/80 βTCP, 21 HA/79 βTCP, 22 HA/78 βTCP, 23 HA/77 βTCP, 24 HA/76 βTCP or 25 HA/75 βTCP.

The amount of the at least one nanoceramic in the scaffold can vary depending on the properties desired. The scaffold can contain about 10, 15, 20, 25, 26, 27, 28, 29, 30, 31, 32, 33, 34, 35, 40, 45 or about 50 wt % of the at least one nanoceramic. These values can be used to define a range, such as about 25 to about 35 wt % nanoceramic. In some embodiments, the nanoceramic can be uniformly distributed throughout the scaffold.

Exemplary composite scaffolds are described in U.S. Publication No. 2016/0000974 entitled “Composite Matrix for Bone Repair Applications”, which is herein incorporated by reference in its entirety.

The scaffold can also contain at least one insulin-mimetic compound. Locally applied insulin and small molecule insulin-mimetics can enhance fracture healing. Insulin-mimetic agents, such as insulin pathway-stimulating vanadium, zinc, tungsten, selenium, molybdemun, niobium, or manganese compounds, can improve the torsional strength and bone mineral density of regenerated bone. The insulin-mimetic compound can be selected from the group consisting of vanadium, zinc, tungsten, selenium, molybdenum, niobium, manganese compounds, and combinations thereof. For example, the insulin-mimetic compound can be vanadyl acetylacetonate. The organic vanadium salt, vanadyl acetylacetonate (VAC), can increase callus cartilage, vascularity, bone formation, and mechanical properties while reducing healing time in fractures.

The amount of the insulin-mimetic compound in the scaffold can vary depending on the properties desired. The scaffold can contain about 0.01, 0.02, 0.03, 0.04, 0.05, 0.06, 0.07, 0.08, 0.09 or about 0.1 wt % of the insulin-mimetic compound. These values can be used to define a range, such as about 0.03 to about 0.07 wt % of the insulin-mimetic compound.

Exemplary insulin-mimetic compounds, compositions and methods of use are described in U.S. Pat. No. 9,265,794 entitled “Insulin-Mimetics as Therapeutic Adjuncts for Bone Regeneration”, which is herein incorporated by reference in its entirety.

In one embodiment, the present disclosure relates to an osteoconductive scaffold capable of the delivery of an insulin-mimetic, such as vanadium, for fracture healing. The scaffold can further include mesenchymal stem cells. The scaffold can stimulate regeneration of bone tissue and repair bone defects. The scaffold can be used alone or in combination with whole bone marrow, isolated mesenchymal stem cells (MSCs) or combinations thereof. Bone cell differentiation can be stimulated in vitro by adding whole bone marrow or isolated MSCs to the matrices and culturing in appropriate conditions.

In another embodiment, the present disclosure relates to a method for repairing a bone defect including the step of applying a composite scaffold to the bone defect, wherein the scaffold includes (i) a synthetic polymer, wherein the synthetic polymer is formed into a plurality of fibers, (ii) at least one nanoceramic, and (iii) an insulin-mimetic compound, wherein the insulin-mimetic compound is continually released from the scaffold. The scaffold can contain about 0.01 to about 0.1 wt % of the insulin-mimetic compound.

The composite scaffold can be applied to the bone defect in any manner, such as applied directly to the site of a bone fracture to accelerate fracture healing, as part of a surgery or percutaneously injected at or near the bone defect. The scaffold can also be applied in combination with an allograft, an autograft, a xenograft, an alloplastic graft, and/or an orthopedic biocomposite.

The scaffold can be manufactured such that the insulin-mimetic compound can be continuously released after application. The insulin-mimetic compound can released over 7, 8, 9, 10, 11, 12, 13, 14, 15, 16, 17, 18, 19, 20, 21, 22, 23, 24, 25, 26, 27, 28, 29 or about 30 days. These values can be used to define a range, such as about 7 to 28 days. The insulin-mimetic compound can be released at a minimum rate over these days, e.g., 7, 8, 9 days, etc. The rate of release can be at least about 5, 10, 15, 20, 25, 30, 35, 40, 45 or about 50 ng/day. These values can be used to define a range, such as about 30 to about 40 ng/day.

The composite scaffold can contain excess insulin-mimetic compound, such that more than about 50, 60, 70, 80, 90 or about 95% of the insulin-mimetic compound is contained in the scaffold after the insulin-mimetic compound is released at the minimum rate over these days, e.g. 7, 8, 9 days, etc. The composite scaffold can also contain minimal amounts of insulin-mimetic compound such that less than about 45, 40, 35, 30, 25, 20, 15, 10 or about 5% of the insulin-mimetic compound is contained in the scaffold after the insulin-mimetic compound is released at the minimum rate over these days, e.g. 7, 8, 9 days, etc.

In addition, the disclosed composite scaffold may advantageously contain and deliver one or more small molecules (e.g., a small molecule with a low molecular weight (e.g., <1000 Dalton) that is/are adapted to regulate a biologic process. Exemplary small molecules for inclusion in the disclosed composite scaffold may be about 1 nm in size. In further exemplary embodiments of the present disclosure, small molecule(s) to be incorporated into the disclosed composite scaffold may be characterized by a molecular weight of less than 500 Daltons.

Exemplary small molecules for inclusion in the disclosed composite scaffold may be advantageously adapted to bind to one or more specific biological targets, e.g., a specific protein or nucleic acid. The exemplary small molecules may also act as an effector, e.g., altering the activity or function of a biologic target. As is known in the art, small molecules having potential applicability in the disclosed composite scaffold may perform (in whole or in part) a variety of biological functions, e.g., serving as cell signaling molecules and/or as a medicinal drug. The present disclosure is not limited in the nature and/or scope of small molecules that may be included in the disclosed composite scaffold, and such compounds may be natural (such as secondary metabolites) or artificial (such as antiviral drugs) and they may have a beneficial effect against a disease (such as drugs) or may be detrimental (such as teratogens and carcinogens).

Exemplary small molecules for inclusion in the disclosed scaffold are molecules/compounds that support or promote bone growth. By way of example, small molecules such as antibiotics or antivirals (e.g., cephalexin, doxycycline hydrochloride, gentamicin sulfate, minocycline hydrochloride, valacyclovir hydrochloride, chloroguanide hydrochloride), pain relievers (e.g., aspirin, acetanilide, antipyrine, nalbuphine hydrochloride), steroids (such as cholecalciferol, fludrocortisone acetate, fluorometholone, halcinonide, medrysone, triamcinolone diacetate, medroxyprogesterone acetate), psychotropic drugs (e.g., zolpidem, spiperone, olanzapine, inositol, isopropamide iodide, clopamide), decalpenic acid, glabrisoflavone, β-cryptoxanthin, prostaglandins, and other miscellaneous small molecules (e.g., avobenzone, aminohippuric acid, tolaxamide, protoveratrine A, ropinirole tolaxamide, trimebutine maleate, ebselen, tuaminoheptane sulfate) may be incorporated into the disclosed scaffold. [See, e.g., Kevin W -H Lo et al., “The role of small molecules in musculoskeletal regeneration,” Regen Med. 2012, Jull 7(4), 535-549.]

In exemplary embodiments, the small molecule(s) may be added to the composite scaffold, e.g., in the electrospinning process, at a desired concentration. The small molecule(s) are advantageously available for controlled release from the scaffold in situ.

The composite scaffold can treat or repair a bone defect by increasing bone growth, retaining mineralized components in bone, inhibiting release of mineralized components from bone, stimulating osteoblast activity, reducing osteoclast activity, and/or stimulating bone remodeling. The scaffold can also increase the production of collagen, glycosaminoglycans (e.g., chondroitin and dermatan sulfate), osteocalcin or combinations thereof. The increased production of each can be about 1%, 5%, 10%, 15%, 20%, 30%, 40%, 50%, 60%, 70%, 80%, 90% or about 100%. These values can also be used to define a range, such as about 20% to about 50%.

The disclosures of all cited references including publications, patents, and patent applications are expressly incorporated herein by reference in their entirety.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The present disclosure is further defined in the following Examples. It should be understood that these Examples, while indicating preferred embodiments of the disclosed systems and methods, are given by way of illustration only.

### EXAMPLES

### Example 1

### In Vitro Evaluation of an Insulin-Mimetic Composite Scaffold for Bone Tissue Repair

Large bone defects that require the use of allograft bone, allograft bone material or synthetic bone graft augmentation are at risk for delayed osseous integration. A osteoconductive scaffold including insulin-mimetics was tested to treat large bone defects. According to the present disclosure, a vanadyl acetylacetonate-releasing composite scaffold was provided for controlled release of VAC and to support bone ingrowth. In the present example, the performance of the VAC composite for supporting osteogenic activity was studied. More particularly, human mesenchymal stem cells (MSCs) differentiation along both osteogenic and chondrogenic lineages in vitro was evaluated. The use of chondrogenic followed by osteogenic differentiation protocols to mimic endochondral ossification was examined. Differentiation was determined by biochemical markers and gene expression over time.

The fibrous scaffolds were fabricated using an electrospinning technique. Scaffolds consisted of 70 wt % polycaprolactone (PCL), 30 wt % of 20/80 (w/w) hydroxyapatite/β-tricalcium phosphate (HA/TCP) nanoparticles (Composite) and the Composite with 0.05 wt % VAC (VAC Composite). The PCL was dissolved in methylene chloride. HA/TCP and VAC were added to the PCL solution for one hour prior to electrospinning. The dispersion was electrospun using a high voltage of 25 kV and a 7 mL/hr flow rate.

Human MSCs were obtained from whole bone marrow aspirates (Lonza, Inc) and cultured using standard protocols. MSCs were cultured on the scaffolds in:

(i) standard growth medium (GM),

(ii) osteogenic medium (OS) (growth medium supplemented with 10 mM β-glycerolphosphate, 50 μM ascorbic acid, and 100 nM dexamethasone),

(iii) chondrogeneic medium (CCM) (0.01 μg/mL TGF-β 3), and

(iv) combination of chondrogenic medium for 14 days followed by osteogenic medium for up to 14 days (C/O).

Cell proliferation was determined by DNA quantitation (Pico Green Assay, Invitrogen). Differentiation was characterized by alkaline phosphatase (ALP) activity (Sigma-Aldrich), osteocalcin (osteocalcin ELISA, LifeTechnologies), total collagen (hydroxyproline, Sigma-Aldrich) and glycosaminoglycan (GAG, Biocolor) production. qRT-PCR (Qiagen) was used for gene expression. Vanadium concentrations were then determined using a Perkin Elmer Z5100 electrothermal heated graphite atomizer with Zeeman background correction (Perkin Elmer Corp., Norwalk, Conn.). Sample size n=4 was used for the proliferation and differentiation assays. A sample size n=3 was used for qRT-PCR. Results were analyzed using a one-way ANOVA and a posthoc Tukey test. Statistically significant values were defined for p<0.05. All statistical analyses were performed using SPSS Statistics software.

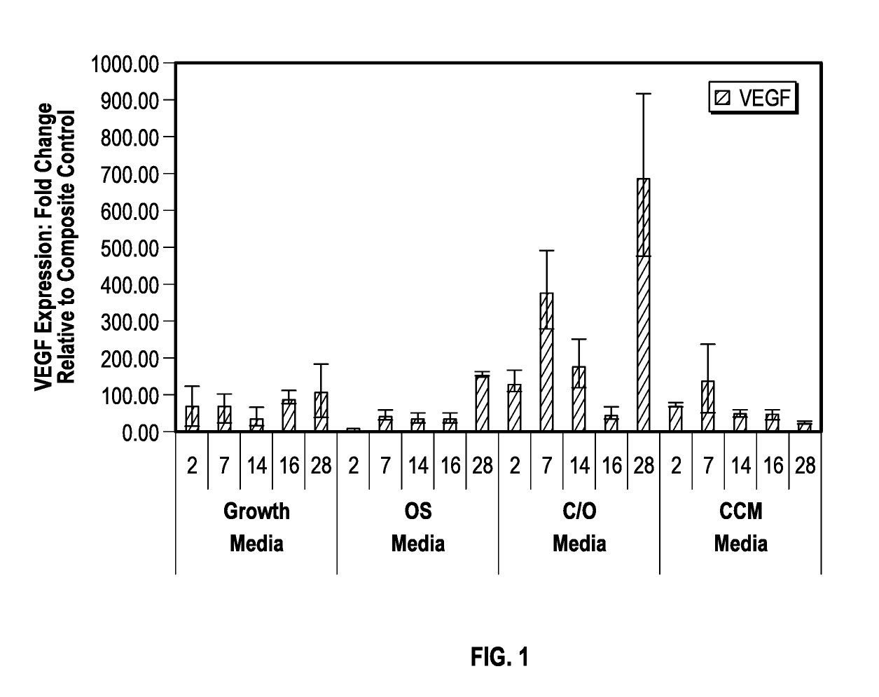

It was observed that the VAC Composite promoted osteogenic/chondrogenic activity. The VAC Composite supported cell growth for up to 28 days in culture. For differentiation, osteogenic and chondrogenic genes were expressed for cells on the VAC Composite over time but most notable was the high expression of VEGF (≧50 fold change relative to the Composite alone at most time points) in all media conditions. FIG. 1 shows that the gene expression for VEGF was increased for cells on VAC Composite relative to Composite control.

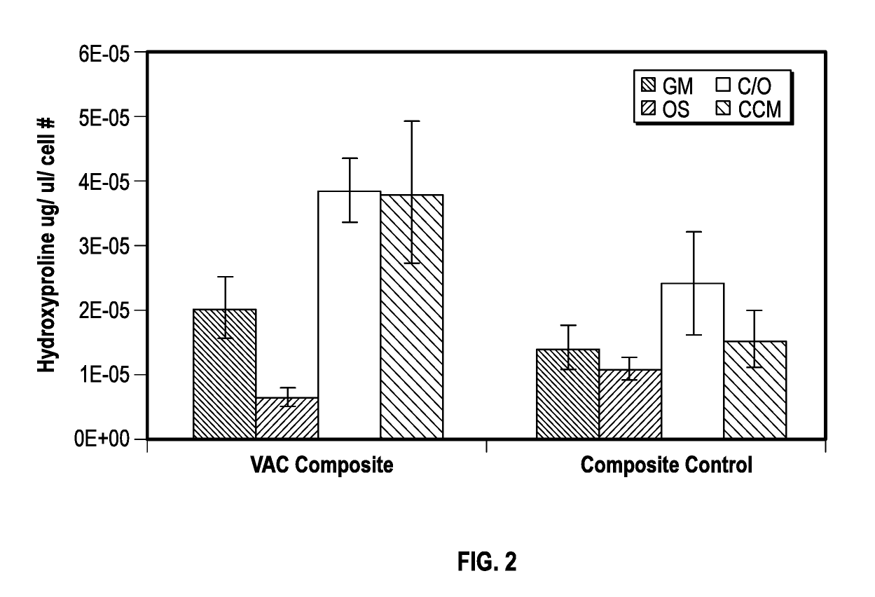

It was also observed that the total collagen production was the highest for cells on the VAC Composite and was higher than the Composite in the C/O and CCM media. FIG. 2 shows the increase in total collagen (hydroxyproline) production at 28 days. In FIGS. 2-4, each set of four bars represents the following medium in order from left to right: (GM) standard growth medium; (OS) osteogenic medium; (C/O) combination of chondrogenic medium for 14 days followed by osteogenic medium for up to 14 days; and (CCM) chondrogeneic medium.

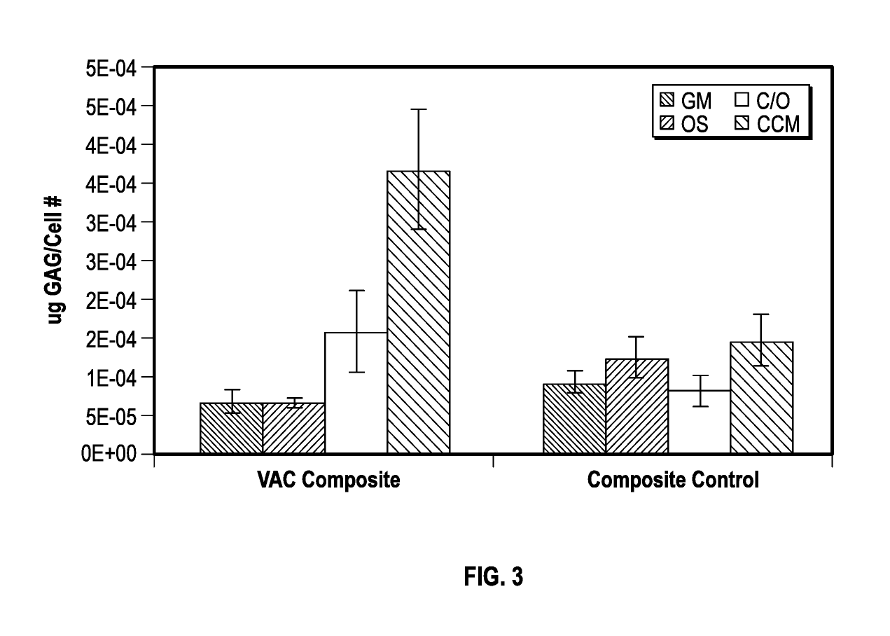

It was also observed that GAG production was the highest for cells on the VAC Composite in the CCM medium and was higher than the Composite in all media conditions. FIG. 3 shows the increased GAG production at 28 days.

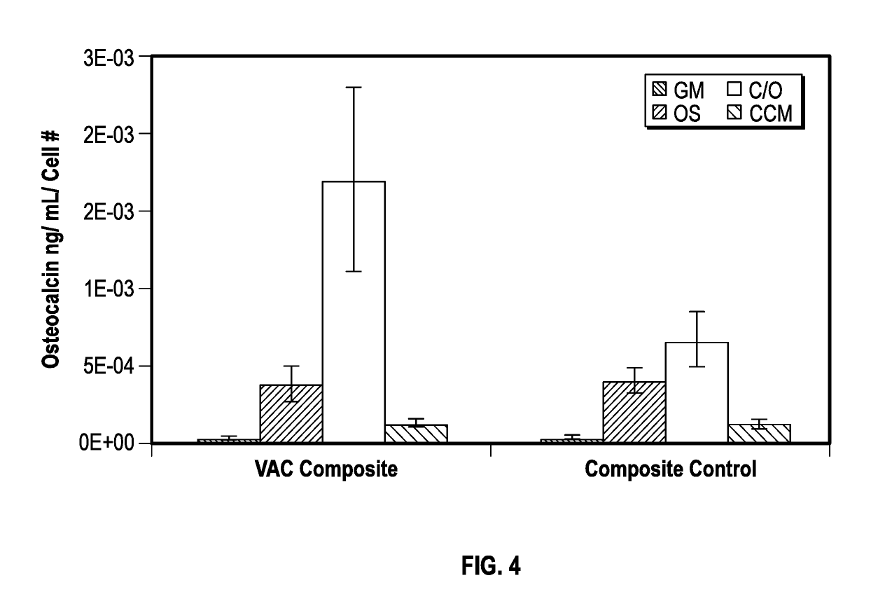

It was observed that for cells on both VAC Composite and Composite, higher levels of ALP and osteocalcin was detected in the C/O medium in comparison to other media. Cells cultured on the VAC Composite in the C/O medium had the highest levels of osteocalcin expression as compared to the other media and the Composite. FIG. 4 shows increased osteocalcin production at 28 days.

Further, it was observed that the VAC composite scaffold maintained continuous release of vanadium at about 35 nanograms/day over the 28 day course. After 28 days, about 48% of the total vanadium was released from the scaffold.

MSC proliferation and differentiation on the VAC Composite shows that these compositions are useful in bone repair. Cells on the VAC Composite showed enhanced production of collagen, and increased expression of ALP and osteocalcin production. In addition, significant VEGF expression was determined on the VAC Composite which suggests it may have the potential to induce angiogenesis. The VAC Composite provides an advantageous delivery vehicle for continuous release of vanadium and a scaffold to support bony ingrowth/integration. The composite scaffold of the present disclosure can also be used to enhance callus formation, mineralization, and overall bone repair.

While this disclosure has been particularly shown and described with reference to the foregoing example thereof, it will be understood by those skilled in the art that various changes in form and details may be made therein without departing from the scope of the invention encompassed by the appended claims.

What is claimed is:

1. A composite scaffold capable of supporting cell and tissue growth comprising:
(i) a synthetic polymer, wherein the synthetic polymer is formed into a plurality of fibers;
(ii) at least one nanoceramic; and
(iii) an insulin-mimetic compound,
wherein the scaffold contains about 0.01 to about 0.1 wt % of the insulin-mimetic compound.

2. The composite scaffold of claim 1, further comprising mesenchymal stem cells.

3. The composite scaffold of claim 1, further comprising whole bone marrow.

4. The composite scaffold of claim 1, wherein the synthetic polymer is selected from the group consisting of polylactic acid, poly L-lactic acid, polyglycolic acid, polylactic co-glycolic acid, poly ε-caprolactone, poly methacrylate co-n-butyl methacrylate, poly dimethyl siloxane, polyethylene oxide and combinations thereof.

5. The composite scaffold of claim 1, wherein the synthetic polymer is poly ε-caprolactone.

6. The composite scaffold of claim 1, wherein the scaffold contains about 65 to about 75 wt % of the synthetic polymer.

7. The composite scaffold of claim 1, wherein the at least one nanoceramic is selected from the group consisting of hydroxy apatite, tricalcium phosphate, biphasic calcium phosphate, calcium carbonate, calcium sulfate, bioactive glass, biphasic bioceramic and combinations thereof.

8. The composite scaffold of claim 1, wherein the at least one nanoceramic is a biphasic bioceramic hydroxyapatite/β-tricalcium phosphate.

9. The composite scaffold of claim 1, wherein the scaffold contains about 25 to about 35 wt % of the at least one nanoceramic.

10. The composite scaffold of claim 1, wherein the at least one nanoceramic is uniformly distributed throughout the scaffold.

11. The composite scaffold of claim 1, wherein the insulin-mimetic compound is selected from the group consisting of vanadium, zinc, tungsten, selenium, molybdenum, niobium, manganese compounds, and combinations thereof.

12. The composite scaffold of claim 1, wherein the insulin-mimetic compound is vanadyl acetylacetonate.

13. The composite scaffold of claim 1, wherein the scaffold contains about 0.03 to about 0.07 wt % of the insulin-mimetic compound.

14. The composite scaffold of claim 1, further comprising one or more small molecules

15. A method for repairing a bone defect comprising applying a composite scaffold to the bone defect, wherein the scaffold includes:
(i) a synthetic polymer, wherein the synthetic polymer is formed into a plurality of fibers;
(ii) at least one nanoceramic; and
(iii) an insulin-mimetic compound, wherein the scaffold contains about 0.01 to about 0.1 wt % of the insulin-mimetic compound, and
wherein the insulin-mimetic compound is continually released from the scaffold.

16. The method of claim 15, further comprising at least one of mesenchymal stem cells and whole bone marrow.

17. The method of claim 15, wherein the insulin-mimetic compound is released over 7 to 28 days.

18. The method of claim 15, wherein the insulin-mimetic compound is released at a rate of at least about 10 ng/day.

19. The method of claim 15, wherein the composite scaffold contains at least about 20% of the insulin-mimetic compound after 7 days after application.

20. The method of claim 15, wherein the composite scaffold further includes one or more small molecules.
